(19) United States
(12) Patent Application Publication
(10) Pub. No.: US 2014/0328826 A1
(43) Pub. Date: Nov. 6, 2014

(54) PRODUCT AND METHOD FOR TREATING KELOID SCARS, HYPERTROPHIC SCARS AND BURN SCARS WITH CONTRACTURE

(71) Applicant: COLE Research & Design, Inc., Jackson, MS (US)

(72) Inventor: Jeptha Newton Cole, Jackson, MS (US)

(73) Assignee: COLE Research & Design, Inc., Jackson, MS (US)

(21) Appl. No.: 14/265,141

(22) Filed: Apr. 29, 2014

Related U.S. Application Data
(60) Provisional application No. 61/818,328, filed on May 1, 2013.

(51) Int. Cl. A61K 38/47; A61K 31/728

(52) U.S. Cl. CPC A61K 38/47; A61K 31/728
USPC 424/94.62; 514/54

(57) ABSTRACT

A method of treating keloid scars, hypertrophic scars or burn scars with contracture includes administering low molecular weight hyaluronic acid to the keloid scar, hypertrophic scar or burn scar with contracture.

### BACKGROUND

Keloid scars are proliferative dermal growths that develop after skin injury. These benign dermal fibroproliferative tumors are made of type I and type Ill collagen, and occur in 5-15% of wounds, with an average age of onset between 10 to 30 years. Furthermore, they occur 15 times more frequently in persons with highly pigmented skin, than in persons of less pigmentation.

Keloid scars can range from mildly cosmetically disfiguring to severely debilitating. Unlike hypertrophic scars, the scar tissue extends beyond the borders of the original wound. These unsightly, lumpy scars can form on any part of the body, and can grow quite large; in extreme cases they grow to the size of tennis balls. Additionally, keloid scars can become inflamed and very painful. In these cases, inflammation develops and the pain is typically not alleviated until the inflammation subsides. A keloid scar in an area that is continually irritated, for example near the waistline, can cause persistent pain, with the keloid scar enlarging and hardening over time. In those affected by keloid scar formation, should a surgical procedure become necessary, for example removal of a skin cancer, the excision itself serves as the injury that stimulates keloid scar formation. Examples of keloid scars are shown in FIG. 3A.

Hypertrophic scars tend to be white or pink in color, with firm tissue limited to the original wound border. Hypertrophic scars may be raised or flat, and are characterized by fewer, more organized collagen fibers than keloid scars. Burn scar with contracture refers to the tightening of the skin after a second or third degree burn. When skin is burned, the surrounding skin begins to pull together, resulting in a contracture. The scar can result in restriction of movement around the injured area.

The traditional first line treatment for keloid scars is a steroid injection, typically KENELOG®, into the fibrous keloid scar mass. Although the steroid injection offers an anti-inflammatory effect thought to reduce further spread and growth of the keloid scar, it provides only a temporary growth reduction requiring repeated injections with decreasing effectiveness. Other treatment options include an immune response modifier medication, for example Imiquimod, pressure dressings to reduce inflammation, topical chemotherapy drugs, laser light therapy, pulsed-dye laser therapy, radiation therapy, surgical excision and silicone gel sheeting application. Reoccurrence is frequent with most of these therapies. Topical silicone gel sheeting application has been shown to prevent keloid scar formation in some cases after excision, however silicone gel sheets need to worn constantly, leading to issues with patient compliance.

Hyaluronic acid is an anionic, non-sulfated, linear unbranched polymer of disaccharides composed of D-glucoronic acid and N-acetyl-D-glucosamine, linked via alternating β-1,4 and β-1,3 glycosidic bonds. Hyaluronic acid is typically classified as high molecular weight hyaluronic acid when the molecular weight is 5×10⁵ Da (500 KDa) or greater, or low molecular weight hyaluronic acid, when the molecular weight is less than 5×10⁵ Da (500 KDa); low molecular weight hyaluronic acid is also referred to as hyaluronan fragments. The classification of hyaluronic acid as high molecular weight and low molecular weight has been inconsistent in the literature.

High molecular weight hyaluronic acid is widely distributed in the human body and is part of the extracellular matrix. It is found abundantly in the skin and participates in tissue repair. High molecular weight hyaluronic acid is also used as a cosmetic dermal filler, by injection into subdermal spaces for the treatment of wrinkles (see, for example, Juvederm® and Restylane®). High molecular weight hyaluronic acid is also used for non-surgical management of joint osteoarthritis, for example by injection into the knee, but other joints have also been treated. However, injection of low molecular weight hyaluronic acid has been shown to lead to inflammation, resulting in symptoms that mimic arthritis.

Hyaluronidases are a family of enzymes that degrade hyaluronic acid, by hydrolysis. Hyaluronidase is known as a “spreading substance”, and has been injected with other medications to improve their absorption. Hyaluronidase has also been used in ophthalmic surgery, in combination with local anesthetics, to improve the uptake of the anesthetic. More recently, hyaluronidase has been used to breakdown high molecular weight hyaluronic acid that has been previously over-injected as dermal fillers.

### SUMMARY

In a first aspect, the present invention is a method of treating keloid scars, hypertrophic scars or burn scars with contracture, comprising administering low molecular weight hyaluronic acid to the keloid scar, hypertrophic scar or burn scar with contracture.

In a second aspect, the present invention is a pharmaceutical composition for treating keloid scars, hypertrophic scars or burn scars with contracture, comprising low molecular weight hyaluronic acid, and at least one pharmaceutically acceptable carrier and/or excipient. The pharmaceutical composition is in unit dosage form.

In a third aspect, the present invention is a pharmaceutical composition for treating keloid scars, hypertrophic scars or burn scars with contracture, comprising high molecular weight hyaluronic acid, hyaluronidase, and at least one pharmaceutically acceptable carrier and/or excipient. The pharmaceutical composition is in unit dosage form.

### DEFINITIONS

High molecular weight hyaluronic acid is hyaluronic acid having a molecular weight of 5×10⁵ Da (500 KDa) or greater. This term also includes salts (such as sodium hyaluronate) and esters thereof.

Low molecular weight hyaluronic acid is hyaluronic acid having a molecular weight of less than 5×10⁵ Da (500 KDa).

### BRIEF DESCRIPTION OF THE DRAWINGS

FIGS. 1A and 1B are two views of a keloid scar of a first patient about 33 days after treatment.

FIGS. 2A and 2B are two views of a keloid scar of a second patient before treatment.

FIGS. 2C and 2D are two views of the keloid scar of the second patient about 13 days after treatment.

FIGS. 3A and 3B are two views of a keroid scar of third patient before treatment.

FIG. 3C is a view of h keloid scar of the third patient about 41 days after treatment.

FIGS. 4A and 4B are views of a first keloid scar of a fourth patient before treatment and about 22 days after treatment, respectively.

FIG. 5A and 5B are views of a second keloid scar of a fourth patient before treatment and about 22 days after treatment, respectively.

FIG. 6 is an illustration of a unit dosage form.

FIG. 7 is an illustration of another unit dosage form.

### DETAILED DESCRIPTION

The present invention makes use of the discovery that low molecular weight hyaluronic acid is effective to soften and shrink keloid scars, when injected into the keloid scar. The keloid scars often shrink a volume of 50% or more, and become soft and collapsible. Typically, only a single injection is necessary to be effective over the course of 2 to 3 weeks. Further injections may be used to further reduce the size of the keloid scar, or further soften the keloid scar. Alternatively, the low molecular weight hyaluronic acid is injected with hyaluronidase. Similarly, the low molecular weight hyaluronic acid is effective to soften and shrink hypertrophic scars, and effective to soften burn scars with contracture.

When skin or tissue becomes injured, fibrocytes migrate to the tissue injury areas where they synthesize and secrete substances and provide the necessary media for wound repair. Fibrocytes also increase the response to receptors in the injured area making them more responsive to the secreted substances. Patients having keloid scars typically have twenty times more fibrocytes than other people, which is believed to make them more susceptible to the substances that attract scarring components and keloid scar development. Low molecular weight hyaluronic acid is believed to be able to induce inflammation and cell injury by stimulating both antigens and receptors; however, it is also believed to inhibit the ability of high molecular weight hyaluronic acid and interleukines to promote fibrocyte differentiation. This provides a possible explanation for the effects of the low molecular weight hyaluronic acid on keloid scars. A similar mechanism is believed to occur with hypertrophic scars and burn scars with contracture.

Low molecular weight hyaluronic acid has a molecular weight of less than 1×10⁶ Da (1000 KDa), and may be prepared by reaction of hyaluronidase with high molecular weight hyaluronic acid. For example, high molecular weight hyaluronic acid (for example, HYALGAN®, sodium hyaluronate, Sanofi-Aventis U.S. LLC, Bridgewater, N.J.) may be mixed with hyaluronidase (for example, HYLENEX® recombinant human hyaluronidase, Halozyme Therapeutics, Inc., San Diego, Calif.), to prepare low molecular weight hyaluronic acid. For example, 1.0 to 10 mg of high molecular weight hyaluronic acid may be mixed with 1.0 to 100 units of hyaluronidase, for example by mixing between two attached syringes. Alternatively, low molecular weight hyaluronic acid may be prepared free of hyaluronidase by fixing the hyaluronidase to a substrate before reaction with the high molecular weight hyaluronic acid. Furthermore, the low molecular weight hyaluronic acid may be separated in to molecular weight fractions (for example, by size exclusion chromatography) any of which may be used individually or in combination, and optionally including hyaluronidase. See, for example, the table below. Also possible is a molecular weight fraction of low molecular weight hyaluronic acid of 150 KDa to 250 KDa.

TABLE
Molecular weight fractions of low
molecular weight hyaluronic acid
200 KDa to 499 KDa
100 KDa to 200 KDa
50 KDa to 100 KDa
10 KDa to 50 KDa
5.0 KDa to 10 KDa
Less than 5.0 KDa

A pharmaceutical composition may contain pharmaceutically acceptable carriers and/or excipients in addition to the low molecular weight hyaluronic acid and optionally hyaluronidase. Examples include buffers (such as monobasic sodium phosphate and dibasic sodium phosphate), water, and salts (such as sodium chloride). Preferably, the low molecular weight hyaluronic acid and/or the hyaluronidase are the sole active ingredients in the pharmaceutical composition. Preferably, the pharmaceutical composition is sterile.

The pharmaceutical composition may be administered directly to the keloid scar, hypertrophic scar or burn scar with contracture by injection directly into the scar. Alternatively, the pharmaceutical composition may be administered topically, for example with a hollow microneedle application, by microneedle dermabrasion, by hydroinjection, or using a transdermal preparation. Examples include Microchannel Skin System, Patient pack 9990P (3M, St. Paul, Minn.), a pre-treatment for treatment of skin with medication which can be used by a patient to penetrate the surface of the skin with minimal to no pain or discomfort on application.

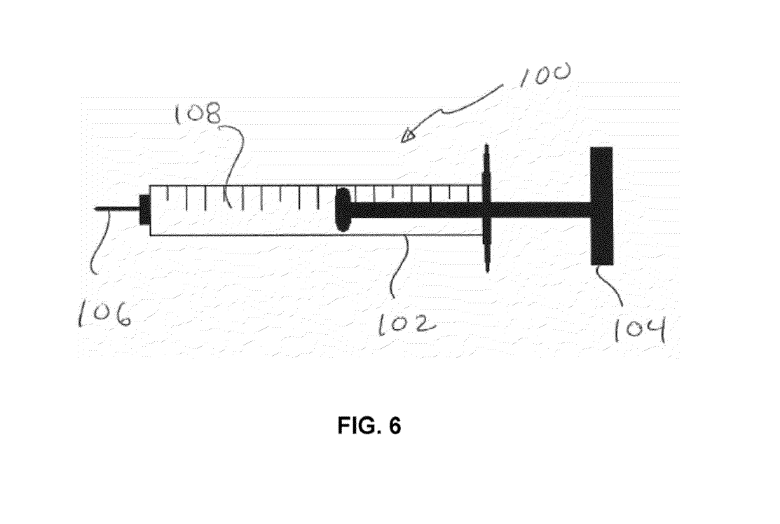

The low molecular weight hyaluronic acid may be provided in a premeasured pharmaceutical composition, for example as single dose, sterile and ready for injection or application. For example, low molecular weight hyaluronic acid may be provided in a premeasured syringe, as illustrated in FIG. 6. As shown, this unit dosage form 100, includes a syringe body, 102, a syringe plunger, 104, and a needle, 106, and a sterile pharmaceutical composition, 108, containing low molecular weight hyaluronic acid in an amount suitable for injection into a single patient, for example 0.05 to 3.0 cc (such as 0.10, 0.20, 0.30, 0.40, 0.50, 0.60, 0.70, 0.80, 0.90, 1.0, 1.1, 1.2, 1.3, 1.4, 1.5, 2.0 and 2.5 cc) of a pharmaceutical composition containing 0.1 to 6.0 mg (such as 0.2, 0.3, 0.4, 0.5, 0.6, 0.7, 0.8, 0.9, 1.0, 1.2, 1.4, 1.5, 1.6, 1.8, 2.0, 2.5, 3.0, 3.5, 4,0, 4.5, 5.0 and 5.5 mg) of low molecular weight hyaluronic acid. Optionally, the pharmaceutical composition may contain 1.0 to 50 units (such as 1.0, 2.0, 3.0, 4.0, 5.0, 7.5, 10, 15, 20, 25, 30, 35, 40 or 45 units) of hyaluronidase.

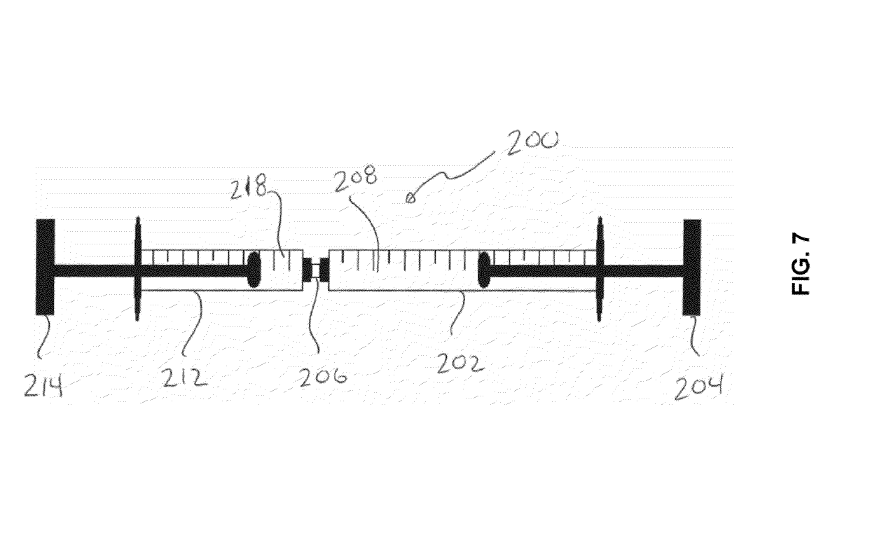

Another unit dosage form is illustrated in FIG. 7. As shown, this unit dosage form, 200, includes a first syringe body, 202, a first syringe plunger, 204, and high molecular weight hyaluronic acid, 208, optionally together with pharmaceutically acceptable carriers and/or excipients, in a premeasured volume of 0.05 to 1.5 cc (such as 0.10, 0.20, 0.30, 0.40, 0.50, 0.60, 070, 0.80, 0.90, 1.0, 1.1, 1.2, 1.3 or 1.4 cc) containing 0.1 to 3 mg (such as 0.2, 0.3, 0.4, 0.5, 0.6, 0.7, 0.8, 0.9, 1.0, 1.2, 1.4, 1.5, 1.6, 1.8, 2.0 or 2.5 mg) of high molecular weight hyaluronic acid, preferably sterile. Also included in a second syringe body, 212, and second syringe plunger, 214, and hyaluronidase, 218, optionally together with pharmaceutically acceptable carriers and/or excipients, in a sterile premeasured amount of 1.0 to 50 units (such as 1.0, 2.0, 3.0, 4.0, 5.0, 7.5, 10, 15, 20, 25, 30, 35, 40 or 45 units) of hyaluronidase. The first and second syringe bodies are connected via a coupler, 206. Just prior to use, the two fluids are mixed by moving the two plungers back and forth, for the low molecular weight hyaluronic acid. Once completely mixed, the syringes may be de-coupled and a needle for injection may be attached, for injection into the keloid scar or scars of a patient. Similarly, both high molecular weight hyaluronic acid and hyaluronidase may be provided as a kit or other form, ready to be mixed, for example by the breaking of a seal or separator between the two reagents.

Keloid scars may be any size, but are typically in the range of about 3 mm×3 mm×3 mm, to about 150 mm×25 mm×25 mm. One or more intralesional injections may be made in each keloid scar, but preferably one injection per cubic centimeter (1 cc or 1000 mm³) of scar tissue is preferred. If a keloid scar is injected multiple times at locations that are too close together, then the injected pharmaceutical composition may leak out a prior injection hole. Preferably, 0.1 to 0.5 cc (such as 0.10, 0.15, 0.20, 0.25, 0.30, 0.35, 0.40 or 0.45 cc) of a pharmaceutical composition is administered per injection. Dosing for the treatment of hypertrophic scars and burn scars with contracture is the same as for keloid scars.

After administration of low molecular weight hyaluronic acid and optionally hyaluronidase, to a patient in need thereof, the keloid scar will begin to shrink and soften, typically over the course of 1 to 3 weeks. In some cases it may be necessary to administer the low molecular weight hyaluronic acid and optionally hyaluronidase a second or third time, with the administrations taking place every 1 to 3 weeks. In most cases, a soft bag of skin will remain. Similarly, hypertrophic scars will begin to shrink and soften, also typically over a course of 1 to 3 weeks. Burn scars with contracture will also begin to soften, but depending on the size, may require more time or treatments.

### EXAMPLES

### Example 1

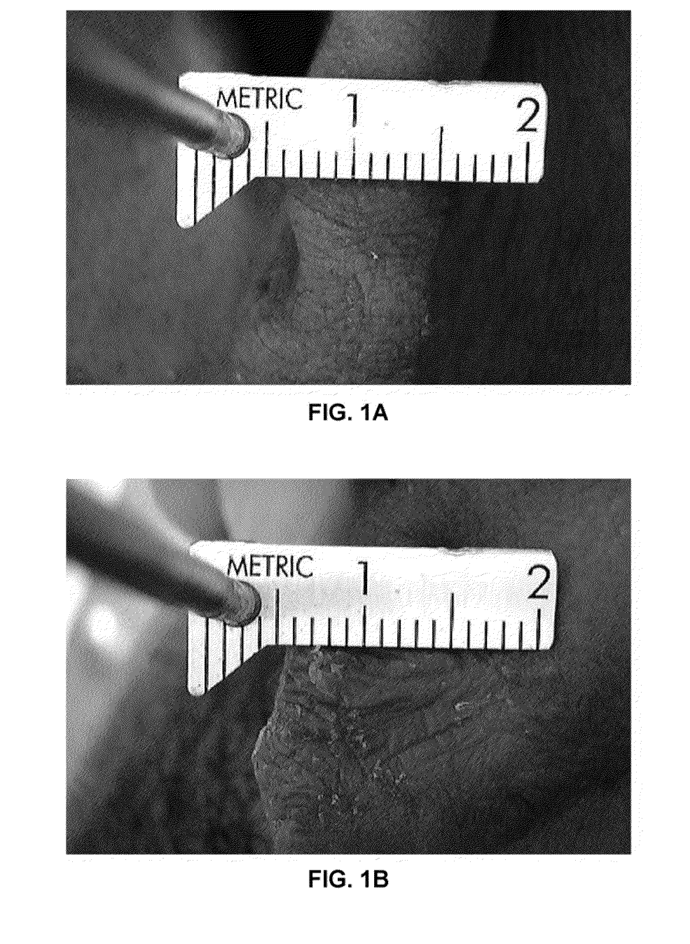

A first patient had a hard keloid scar on the right earlobe measuring 10 mm×5 mm×10 mm. A pharmaceutical composition was prepared by mixing 1.0 mg of high molecular weight hyaluronic acid (0.1 cc of HYALGAN®, sodium hyaluronate) with 7.5 units of hyaluronidase (HYLENEX® recombinant human hyaluronidase), which was injected into the keloid scar. Within 1 to 2 weeks post injection a noticeable reduction is size and hardness of the keloid scar had occurred. Thirty-eight days post injection the keloid scar was almost completely resolved, with an approximately 100% reduction in height, a nearly 40% reduction in length, and a nearly 100% reduction in width (see FIGS. 1A and 1B). Erythema and tenderness of the keloid scar was also resolved.

### Example 2

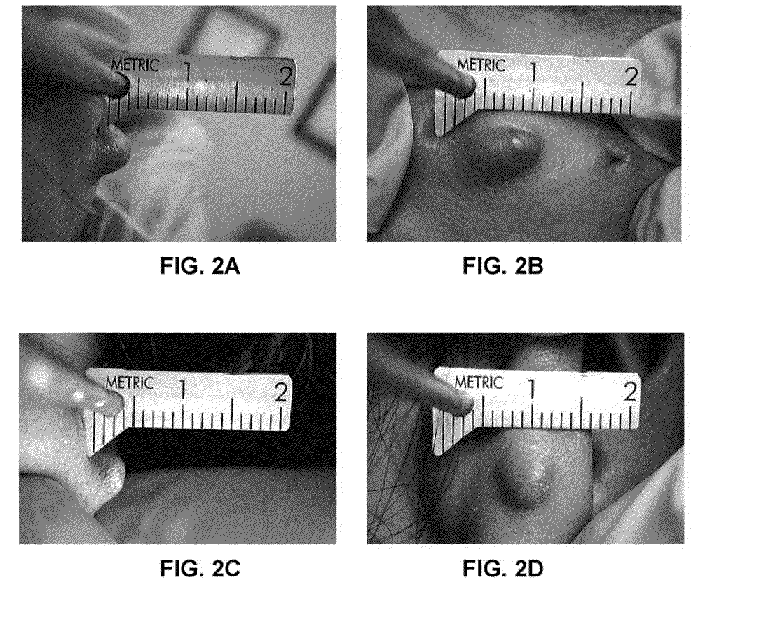

A second patient had a very dense keloid scar on the right earlobe measuring 8.5 mm×6.5 mm×5 mm (see FIGS. 2A and 2B). A pharmaceutical composition was prepared as in Example 1, and injected into the keloid scar. Thirteen days post injection, the keloid scar had a noticeable overall reduction in size and density, with an approximately 24% reduction in length, 30% reduction in height, and 8% reduction in width (see FIGS. 2C and 2D). This patient also had a very dense small left earlobe keloid scar that measured 2×2×2 mm, which was treated at the same time; thirteen days post injection resulted in tissue softening and an approximately 50% reduction in size.

### Example 3

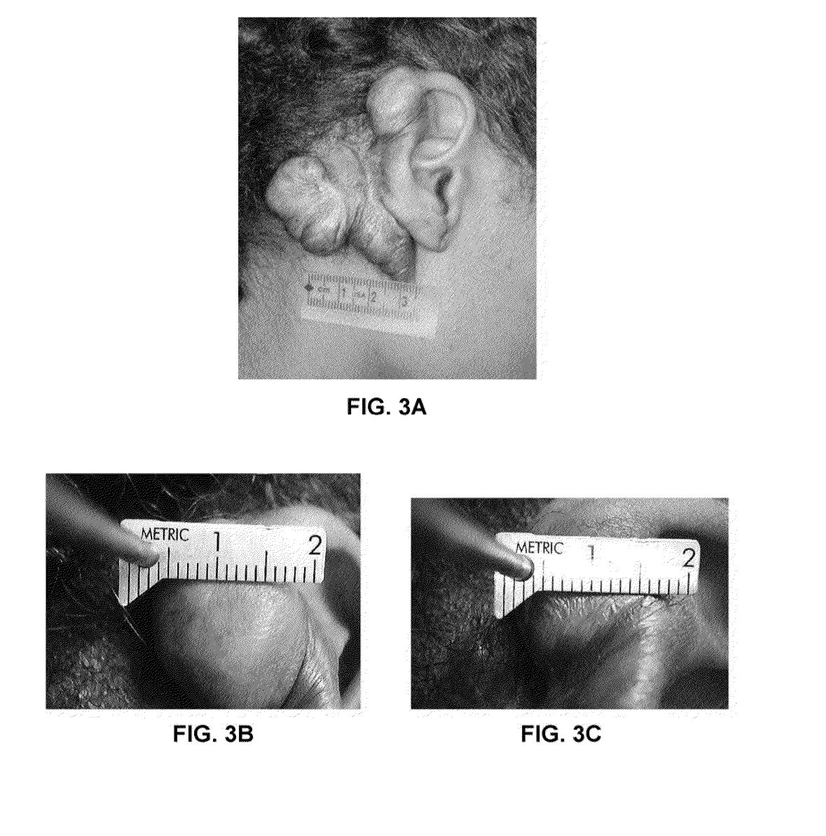

A third patient had a large dense keloid scar behind the right ear measuring 68 mm×45 mm×16 mm (see FIGS. 3A and 3B). The patient also reported a sharp, piercing pain from the keloid scar. A pharmaceutical composition was prepared as in Example 1, and injected into the keloid scar. Only a small reduction in size of the keloid scar occurred within 2 weeks, so the keloid scar was treated again. Forty-one days post injection the piercing pain had resolved, and the keloid scar was substantially reduced in the size and density: the scar had a negligible reduction in length, 31% reduction in height, and 11% reduction in width (see FIG. 3C).

### Example 4

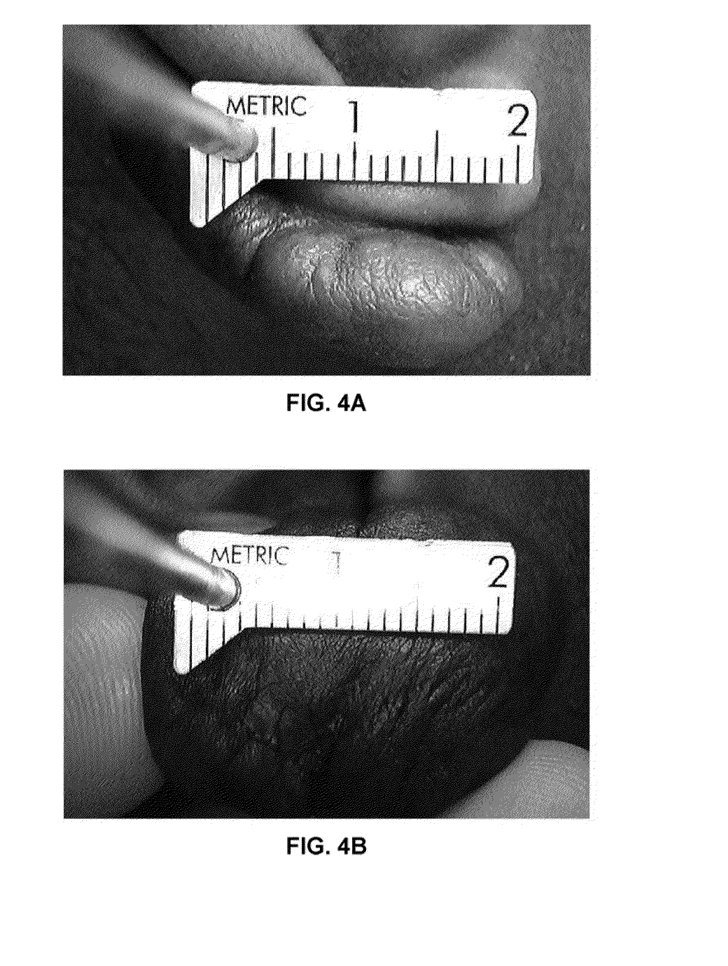

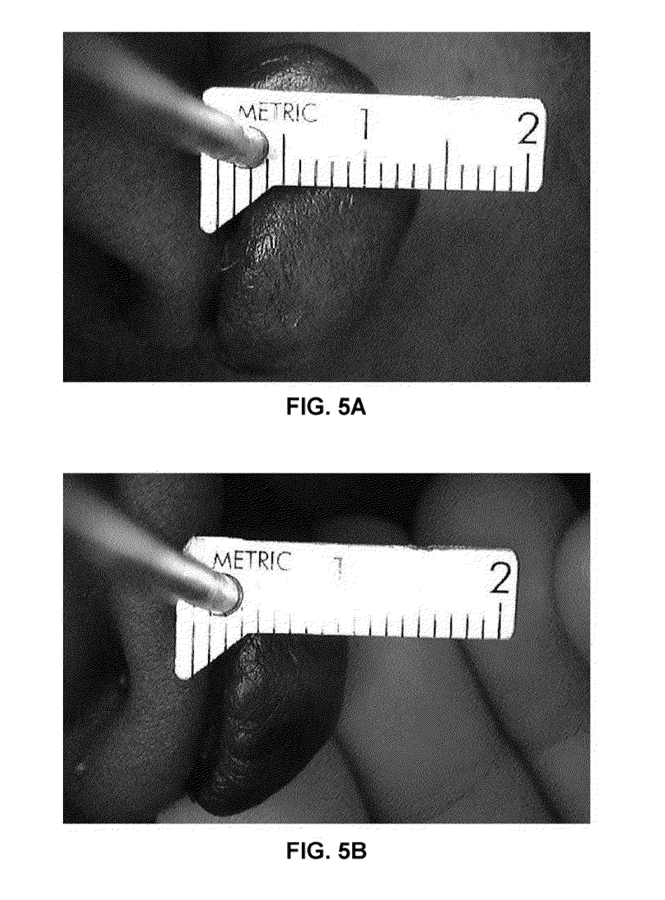

A fourth patient had two keloid scars, one on each earlobe. A pharmaceutical composition was prepared as in Example 1, and injected into each of the keloid scars. Twenty-two days post injection, the keloid scar on the right earlobe was reduced to a floppy skin sack (see FIGS. 4A and 4B, showing the keloid scar at the time of injection and after 22 days, respectively). Similarly, the very dense keloid scar on the left earlobe (originally measuring 31 mm×9 mm in length and width, respectively; see FIG. 5A), became much softer and was significantly reduced in length and width (see FIG. 5B). Some itching was reported by the patient.

Analysis

Four patients had a total of 6 keloid scars treated over a 3 to 6 week period. All four patients responded to the treatment. Overall, there was significant reduction in keloid scar size, with an average of approximately a 22% reduction in length, a 37% reduction in width, and a 45% reduction in height. Even more striking was great reduction in density and hardness of the keloid scar tissue, leaving a soft pliable skin sack after treatment.

Only a few mild adverse reactions were reported. Two patients reported very mild itching from the keloid scars. Some stinging from the injection was also reported. No significant pain or swelling was reported.

### REFERENCES

C. J. Chike-Obi, P. D. Cole, and A. E. Brissett “Keloids: Pathogenesis, Clinical Features, and Management” Seminars In Plastic Surgery 23, (3) 178-184 (2009).

Gauglitz, et al. “Hypertrophic Scarring and Keloids: Pathomechanisms and Current and Emerging Treatment Strategies” Mol. Med. 17 (1-2) 113-125, (2011).

S. M. Silverstein, et al. “Hyaluronidase in Ophthalmology” The Journal of Applied Research 12 (1) 1-13 (2012).

H. J. Brody “Use of Hyaluronidase in the Treatment of Granulomatous Hyaluronic Acid Reactions or Unwanted Hyaluronic Acid Misplacement” Dermatol Surg 31:8 Part 1: 893-897 (2005).

A. S. Maharjan, et al “High and Low Molecular Weight Hyaluronic Acid Differentially Regulate Human Fibrocyte Differentiation” PLoS ONE 6 (10) e26078 (2011).

Blakaj A. Bucala R. “Fibrocytes in health and disease” Fibrogenesis in Tissue & Repair 5(Suppl 1):S6 (2012).

Kao H K, Chen B, Murphy G F, Li Q. Orgill D P, Guo L “Peripheral blood fibrocytes: enhancement of wound healing by cell proliferation, re-epithelialization, contration, and angiogensis” Ann Surg. 254(6):1066-74 (2011).

Naylor M C, Lazar D A, Zamora I J, Mushin O P, Yu L, Brissett A E, Olutoye O O “Increased in vitro differentiation of fibrocytes from keloid patients is inhibited by serum amyloid P” Wound Repair Regen. 20(3):277-83 (2012).

Yang J A, Kim E S, Kwon J H, Kim H, Shin J H, Un S H, Choi K Y, Hahn S K “Transdermal delivery of hyaluronic acid-human growth hormone conjugate” Biomaterials 33(25):5947-54 (2012).

Campo G M, Avenoso A, D'Ascola A, Scuruchi M, Prestipino V, Nastasi G. Calatroni A. Campo S “Adenosine A2A receptor activation and hyaluronan fragment inhibition reduce inflammation in mouse articular chondrocytes stimulated with interlukin-1B” FEBS J. 279(12):2120-33 (2012).

“Hyaluronan” en.wikipedia.org/w/index.php?oldid=534121954 (downloaded Apr. 29, 2013).

“Hyaluronidase” en.wikipedia.org/w/index.php?oldid=532714103 (downloaded Apr. 29, 2013).

Berman B, Harrison-Balestra C. Ramirez C. et al. “Treatment of Keloid Scars Post-Shave Excision With Imiquimod 5% Cream: A Prospective, Double-Blind, Placebo-Controlled Pilot Study” Journal Of Drugs In Dermatology 8(5):455-458 (2009).

B. Berman, F. Flores “The treatment of hypertrophic scars and keloids” European Journal of Dermatology 8(8):591-6 (1998).

1. A method of treating keloid scars, hypertrophic scars or burn scars with contracture, comprising administering low molecular weight hyaluronic acid to the keloid scar, hypertrophic scar or burn scar with contracture.

2. The method of claim 1, wherein the administering comprises administering a pharmaceutical composition comprising:
the low molecular weight hyaluronic acid, and
at least one pharmaceutically acceptable carrier and/or excipient.

3. The method of claim 1, wherein the low molecular weight hyaluronic acid is administered in an amount of 0.1 to 3.0 mg.

4. (canceled)

5. The method of claim 1, wherein low molecular weight hyaluronic acid is administered a plurality of time to the keloid scar, hypertrophic scar or burn scar with contracture.

6. The method of claim 1, wherein the low molecular weight hyaluronic acid is administered a plurality of times, wherein administration is carried out once per every 0.2 to 2.0 cm³ of scar tissue.

7. (canceled)

8. The method of claim 1, wherein the administering comprises administering a composition comprising:
the low molecular weight hyaluronic acid,
hyaluronidase, and
optionally a pharmaceutically acceptable carrier and/or excipient.

9. The method of claim 1, wherein the low molecular weight hyaluronic acid is prepared by mixing high molecular weight hyaluronic acid and hyaluronidase.

10. (canceled)

11. The method of claim 1, wherein the hyaluronidase is present in an amount of 5.0 to 15 units.

12. The method of claim 1, wherein the administering comprises administering a pharmaceutical composition comprising:
the low molecular weight hyaluronic acid,
hyaluronidase, and
at least one pharmaceutically acceptable carrier and/or excipient,
wherein the low molecular weight hyaluronic acid is present in an amount of 0.5 to 1.5 mg,
the hyaluronidase is present in an amount of 5.0 to 15 units, and
the total volume of pharmaceutical composition administered is 0.10 to 0.30 cm³.

13. (canceled)

14. A pharmaceutical composition for treating keloid scars, hypertrophic scars or burn scars with contracture, comprising:
low molecular weight hyaluronic acid, and
at least one pharmaceutically acceptable carrier and/or excipient,
wherein the pharmaceutical composition is in unit dosage form.

15. The pharmaceutical composition of claim 14, further comprising hyaluronidase.

16. The pharmaceutical composition of claim 14, wherein the unit dosage form comprises a syringe.

17. The pharmaceutical composition of claim 14, wherein wherein the low molecular weight hyaluronic acid is prepared by mixing high molecular weight hyaluronic acid and hyaluronidase.

18. (canceled)

19. The pharmaceutical composition of claim 14, wherein the low molecular weight hyaluronic acid is present in an amount of 0.5 to 1.5 mg.

20. (canceled)

21. The pharmaceutical composition of claim 14, wherein the hyaluronidase is present in an amount of 5.0 to 15 units.

22. The pharmaceutical composition of claim 14, comprising:
the low molecular weight hyaluronic acid,
hyaluronidase, and
the at least one pharmaceutically acceptable carrier and/or excipient,
wherein the low molecular weight hyaluronic acid is present in an amount of 0.5 to 1.5 mg, and
the hyaluronidase is present in an amount of 5.0 to 15 units.

23. (canceled)

24. A kit for treating keloid scars, hypertrophic scars or burn scars with contracture, comprising:
high molecular weight hyaluronic acid,
hyaluronidase, and
at least one pharmaceutically acceptable carrier and/or excipient,
wherein the pharmaceutical composition is in unit dosage form.

25-29. (canceled)

30. The kit of claim 24, comprising:
the high molecular weight hyaluronic acid,
the hyaluronidase, and
the at least one pharmaceutically acceptable carrier and/or excipient,
wherein the high molecular weight hyaluronic acid is present in an amount of 0.5 to 1.5 mg, and
the hyaluronidase is present in an amount of 5.0 to 15 units.

31. The kit of claim 24, wherein the unit dosage form comprises a first syringe containing the high molecular weight hyaluronic acid, and a second syringe containing the hyaluronidase.

32. The method of claim 1, wherein the method is a method of treating keloid scars.

33-37. (canceled)
